(19) United States
(12) Patent Application Publication
(10) Pub. No.: US 2025/0295890 A1
(43) Pub. Date: Sep. 25, 2025

(54) BI-DIRECTIONAL STEERABLE CATHETER WITH AUTO LOCK FEATURE

(71) Applicant: Boston Scientific Scimed, Inc., Maple Grove, MN (US)

(72) Inventors: Zachary Edward Nelson, Minnetonka, MN (US); Nicholas Lee Tassoni, Andover, MN (US); Sarah Tian-Hong Hua, Plymouth, MN (US)

(73) Assignee: BOSTON SCIENTIFIC SCIMED, INC., MAPLE GROVE, MN (US)

(21) Appl. No.: 19/086,904

(22) Filed: Mar. 21, 2025

Related U.S. Application Data
(60) Provisional application No. 63/569,615, filed on Mar. 25, 2024.

(51) Int. Cl. A61M 25/01

(52) U.S. Cl. CPC A61M 25/0147

(57) ABSTRACT

A bi-directional steerable catheter includes a handle and a steerable elongate sheath extending distally from the handle. A rotatable knob may be actuated to cause a distal portion of the steerable elongate sheath to bend in a first direction or a second direction. An auto lock feature holds the rotatable knob at its rotational position when a user releases the rotatable knob. The auto lock feature may include particular geometries, materials and surface roughness parameters.

### CROSS-REFERENCE TO RELATED APPLICATIONS

This application is a continuation of U.S. Patent Application Ser. No. 63/569,615, filed Mar. 25, 2024, entitled “BI-DIRECTIONAL STEERABLE CATHETER WITH AUTO LOCK FEATURE”, which is incorporated by reference herein in its entirety.

### TECHNICAL FIELD

The disclosure relates generally to medical devices and more particularly to mechanisms for steering catheters, sheaths, and/or elongate tubular shafts including an auto lock feature.

### BACKGROUND

A wide variety of intracorporeal medical devices have been developed for medical use, for example, surgical and/or intravascular use. Some of these devices include guidewires, catheters, medical device delivery systems (e.g., for stents, grafts, replacement valves, etc.), and the like. These devices are manufactured by any one of a variety of different manufacturing methods and may be used according to any one of a variety of methods. There is an ongoing need to provide alternative medical devices as well as alternative methods for manufacturing and/or using medical devices.

### SUMMARY

The disclosure relates generally to medical devices and more particularly to mechanisms for steering catheters, sheaths, and/or elongate tubular shafts including an auto lock feature. An example may be found in a bi-directional steerable catheter that includes a handle and an elongate shaft extending distally from the handle. The handle includes an outer surface and a handle recess formed within the outer surface. An axial translation mechanism is disposed within the handle. The axial translation mechanism includes a threaded member slidably disposed within the handle and a rotatable knob disposed about the handle recess and configured to engage the threaded member such that rotation of the rotatable knob relative to the handle causes axial translation of the threaded member within the handle. A first steering wire extends through the elongate sheath from the handle to a distal pull ring and is configured to engage with the threaded member to bend a distal portion of the elongate sheath in a first direction. A second steering wire extends through the elongate sheath from the handle to the distal pull ring and is configured to engage with the threaded member to bend the distal portion of the elongate sheath in a second direction opposite the first direction. The bi-directional steerable catheter is adapted to provide an auto lock feature that holds the rotatable knob at its rotational position when a user releases the rotatable knob while the distal portion of the elongate sheath is bent in the first direction or in the second direction.

Alternatively or additionally, the rotatable knob may define a first distal annular bearing surface and a first proximal annular bearing surface. The handle recess may define a second distal annular bearing surface that is parallel to and adapted to engage the first distal annular bearing surface, and a second proximal annular surface bearing surface that is parallel to and adapted to engage the first proximal annular bearing surface.

Alternatively or additionally, the first distal annular bearing surface may have an average surface roughness of less than about 1.27 micrometers (50 microinches), the first proximal annular bearing surface may have an average surface roughness of less than about 1.27 micrometers (50 microinches), the second distal annular bearing surface may have an average surface roughness of less than about 1.27 micrometers (50 microinches), and the second proximal annular bearing surface may have an average surface roughness of less than about 1.27 micrometers (50 microinches).

Alternatively or additionally, the first distal annular bearing surface may have an average surface roughness that is in a range of about 0.127 micrometers (5 microinches) to about 1.02 micrometers (40 microinches), the first proximal annular bearing surface may have an average surface roughness that is in a range of about 0.127 micrometers (5 microinches) to about 1.02 micrometers (40 microinches), the second distal annular bearing surface may have an average surface roughness that is in a range of about 0.127 micrometers (5 microinches) to about 1.02 micrometers (40 microinches), and the second proximal annular bearing surface may have an average surface roughness that is in a range of about 0.127 micrometers (5 microinches) to about 1.02 micrometers (40 microinches).

Alternatively or additionally, the rotatable knob may have a threaded inner surface adapted to engage the threaded member, and the threaded inner surface may have an average surface roughness of less than about 1.27 micrometers (50 microinches).

Alternatively or additionally, the threaded inner surface may have an average surface roughness that is in a range of about 0.127 micrometers (5 microinches) to about 1.02 micrometers (40 microinches).

Alternatively or additionally, the handle may include a polycarbonate acrylonitrile-butadiene-styrene polymer blend.

Alternatively or additionally, the rotatable knob may include a first dial half defining part of the threaded inner surface, a second dial half defining part of the threaded inner surface and secured to the first dial half, and a graspable boot disposed over the first dial half and the second dial half.

Alternatively or additionally, the first dial half and the second dial half may each include a polycarbonate acrylonitrile-butadiene-styrene polymer blend that includes about five weight percent poly tetrafluoroethylene.

Alternatively or additionally, the outer surface of the housing may include a graspable surface having a surface roughness of greater than 1.91 micrometers (75 microinches).

Alternatively or additionally, the auto lock feature may include a tactile neutral position when a load on the first steering wire is equal to a load on the second steering wire, including when there is no load on either the first steering wire or the second steering wire.

Another example may be found in a bi-directional steerable catheter having a handle including an outer surface and a handle recess formed within the outer surface, the handle recess having an average surface roughness of less than about 1.27 micrometers (50 microinches). An axial translation mechanism is disposed within the handle and includes a threaded member slidably disposed within the handle, and a rotatable knob disposed about the handle recess and configured to engage the threaded member such that rotation of the rotatable knob relative to the handle causes axial translation of the threaded member within the handle, where portions of the rotatable knob contacting the handle recess have an average surface roughness of less than about 1.27 micrometers (50 microinches). An elongate sheath extends distally from the handle. A first steering wire extends through the elongate sheath from the handle to a distal pull ring and is configured to engage with the threaded member to bend a distal portion of the elongate sheath in a first direction. A second steering wire extends through the elongate sheath from the handle to the distal pull ring and is configured to engage with the threaded member to bend the distal portion of the elongate sheath in a second direction opposite the first direction.

Alternatively or additionally, the portions of the rotatable knob contacting the handle recess may include a first distal annular bearing surface and a first proximal annular bearing surface. The handle recess may include a second distal annular bearing surface that is parallel to and adapted to engage the first distal annular bearing surface and a second proximal annular surface bearing surface that is parallel to and adapted to engage the first proximal annular bearing surface.

Alternatively or additionally, the rotatable knob may have a threaded inner surface adapted to engage the threaded member, and the threaded inner surface may have an average surface roughness of less than about 1.27 micrometers (50 microinches).

Alternatively or additionally, the handle may include a polycarbonate acrylonitrile-butadiene-styrene polymer blend.

Alternatively or additionally, the rotatable knob may include a first dial half defining part of the threaded inner surface, a second dial half defining part of the threaded inner surface and secured to the first dial half, and a graspable boot disposed over the first dial half and the second dial half.

Alternatively or additionally, the first dial half and the second dial half may each include a polycarbonate acrylonitrile-butadiene-styrene polymer blend that includes about five weight percent poly tetrafluoroethylene.

Another example may be found in a bi-directional steerable catheter having a handle including an outer surface and a handle recess formed within the outer surface. An axial translation mechanism is disposed within the handle and includes a threaded member slidably disposed within the handle, and a rotatable knob that is disposed about the handle recess and is configured to engage the threaded member such that rotation of the rotatable knob relative to the handle causes axial translation of the threaded member within the handle. The rotatable knob includes a first dial half and a second dial half that together define a first distal annular bearing surface, a first proximal annular bearing surface, and a threaded surface adapted to engage the threaded member. The handle recess includes a second distal annular bearing surface that is parallel to and adapted to engage the first distal annular bearing surface, and a second proximal annular surface bearing surface that is parallel to and adapted to engage the first proximal annular bearing surface. An elongate sheath extends distally from the handle. A first steering wire extends through the elongate sheath from the handle to a distal pull ring and is configured to engage with the threaded member to bend a distal portion of the elongate sheath in a first direction. A second steering wire extends through the elongate sheath from the handle to the distal pull ring and is configured to engage with the threaded member to bend the distal portion of the elongate sheath in a second direction opposite the first direction. The handle includes a polycarbonate acrylonitrile-butadiene-styrene polymer blend. The first dial half and the second dial half each include a polycarbonate acrylonitrile-butadiene-styrene polymer blend that includes about five weight percent poly tetrafluoroethylene.

Alternatively or additionally, the first distal annular bearing surface may have an average surface roughness of less than about 1.27 micrometers (50 microinches), the first proximal annular bearing surface may have an average surface roughness of less than about 1.27 micrometers (50 microinches), the second distal annular bearing surface may have an average surface roughness of less than about 1.27 micrometers (50 microinches), and the second proximal annular bearing surface may have an average surface roughness of less than about 1.27 micrometers (50 microinches).

Alternatively or additionally, the first distal annular bearing surface may have an average surface roughness that is in a range of about 0.127 micrometers (5 microinches) to about 1.02 micrometers (40 microinches), the first proximal annular bearing surface may have an average surface roughness that is in a range of about 0.127 micrometers (5 microinches) to about 1.02 micrometers (40 microinches), the second distal annular bearing surface may have an average surface roughness that is in a range of about 0.127 micrometers (5 microinches) to about 1.02 micrometers (40 microinches), and the second proximal annular bearing surface may have an average surface roughness that is in a range of about 0.127 micrometers (5 microinches) to about 1.02 micrometers (40 microinches).

Alternatively or additionally, the threaded inner surface may have an average surface roughness that is in a range of about 0.127 micrometers (5 microinches) to about 1.02 micrometers (40 microinches).

The above summary of some example embodiments is not intended to describe each disclosed embodiment or every implementation of the disclosure.

### BRIEF DESCRIPTION OF THE DRAWINGS

The disclosure may be more completely understood in consideration of the following detailed description of various embodiments in connection with the accompanying drawings, in which:

FIG. 1 illustrates selected features of an example steerable catheter;

FIG. 2 illustrates selected features of the example steerable catheter of FIG. 1;

FIG. 3 illustrates selected features of the example steerable catheter of FIG. 1;

FIG. 4 illustrates selected features of the example steerable catheter of FIG. 1;

FIG. 5 illustrates selected features of steering the example steerable catheter of FIG. 1 in a first direction;

FIG. 6 illustrates selected features of steering the example steerable catheter of FIG. 1 in a second direction;

FIG. 7 illustrates selected features of the example steerable catheter of FIG. 1;

FIG. 8 illustrates selected features of the example steerable catheter of FIG. 1;

FIG. 9 illustrates selected features of the example steerable catheter of FIG. 1;

FIG. 10 illustrates selected features of the example steerable catheter of FIG. 1;

FIG. 11 illustrates selected features of the example steerable catheter of FIG. 1;

FIG. 12 is a cross-sectional view taken along the line 12-12 of FIG. 11.

While features of the disclosure are amenable to various modifications and alternative forms, specifics thereof have been shown by way of example in the drawings and will be described in detail. It should be understood, however, that the intention is not to limit features of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “extent” and/or “maximum extent” may be understood to mean a greatest measurement of a stated or identified dimension, while the term “minimum extent” may be understood to mean a smallest measurement of a stated or identified dimension. For example, “outer extent” may be understood to mean a maximum outer dimension, “radial extent” may be understood to mean a maximum radial dimension, “longitudinal extent” may be understood to mean a maximum longitudinal dimension, etc. Each instance of an “extent” may be different (e.g., axial, longitudinal, lateral, radial, circumferential, etc.) and will be apparent to the skilled person from the context of the individual usage. Generally, an “extent” or “maximum extent” may be considered a greatest possible dimension measured according to the intended usage. Alternatively, a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to effect the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

In some medical procedures, delivery and/or access sheaths may be routed percutaneously into a body cavity, lumen, and/or treatment site. Navigation through patient vasculature and/or organs may include steering through tortuous anatomy and/or directing a distal end of the delivery and/or access sheath into a body cavity, lumen, and/or treatment site. Examples of medical devices suitable for use in medical procedures, such as but not limited to left atrial appendage closure, aortic valve replacement, mitral valve replacement, septal defect repair, etc., are described herein. Existing medical devices may have certain advantages and/or disadvantages. There is an ongoing need for alternative steerable medical devices for delivering medical implants and/or conducting other treatment procedures.

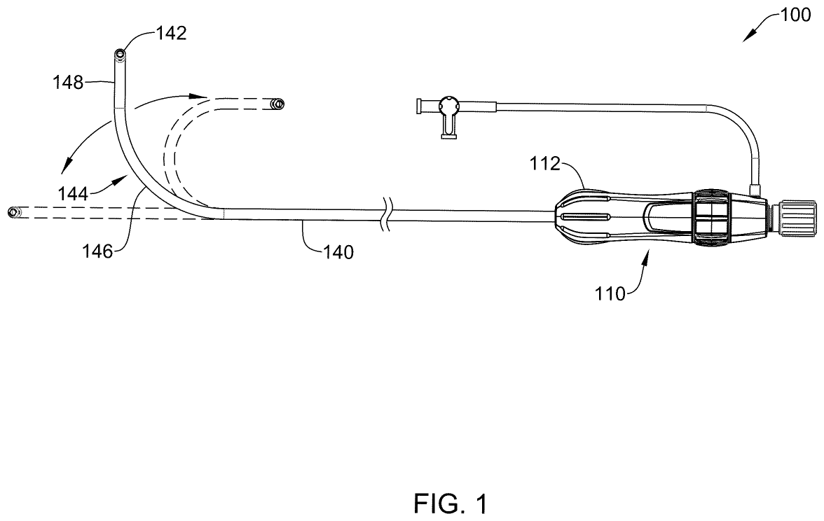

FIG. 1 illustrates selected features of a bi-directional steerable catheter 100. In some instances, the bi-directional steerable catheter 100 may be any one of a variety of catheters, such as an intravascular catheter. Examples of intravascular catheters may include, but are not limited to, balloon catheters, atherectomy catheters, device delivery catheters, drug delivery catheters, diagnostic catheters, and guide catheters. In some cases, the bi-directional steerable catheter 100 may take the form of other suitable guiding, diagnosing, or treating devices (including endoscopic instruments, laparoscopic instruments, etc., and the like) and it may be suitable for use at various locations and/or body lumens within a patient.

The bi-directional steerable catheter 100 may include a handle 110 and an elongate sheath 140 extending distally from the handle 110. In some embodiments, the bi-directional steerable catheter 100 and/or the handle 110 may include a guidewire port, a side port, a fluid flush port, an imaging access port, or other suitable ports, access points, or functional features. The handle 110 may include a handle housing 112. The elongate sheath 140 may extend into and/or through a distal opening in the handle housing 112. In at least some cases, a proximal end of the elongate sheath 140 may be fixedly attached to and/or inside of the handle housing 112. In some cases, a proximal portion of the elongate sheath 140 may include a key element configured to non-rotatably engage one or more lock elements fixedly attached to an inner surface of the handle housing 112 proximal a distal end of the handle housing 112. In some cases, the key element may be bonded to an outer surface of the elongate sheath 140. In some cases, the key clement may be integrally formed with the elongate sheath 140. In some cases, the key element may be welded (e.g., heat weld, sonic weld, vibration weld, etc.) to elongate sheath 140. In some cases, the key element may be melted together with the elongate sheath 140 such that material of the key element is co-mingled with material of the elongate sheath 140 at a molecular level. In some cases, the handle housing 112 may include one or more lock elements fixedly attached to and/or integrally formed with the inner surface of the handle housing 112. In some cases, the one or more lock elements may be formed as ribs or other structural support members configured to increase the rigidity of the handle housing and permit torque transfer between the distal end of the handle housing 112 and the elongate sheath 140. In some cases, the elongate sheath 140 may have a normal or relaxed configuration. The elongate sheath 140 may be self-biased toward, and/or in the absence of any outside forces may return to, the normal or relaxed configuration. Some suitable but non-limiting materials for the handle 110 and/or the handle housing 112 are described below.

In some instances, the elongate sheath 140 may include a soft and/or atraumatic distal tip 142. In some instances, the elongate sheath 140 may include a distal portion 144 having a first curve 146 and a second curve 148, such that the elongate sheath 140 has a preset double curve, in the normal or relaxed configuration. In some instances, the first curve 146 may be preset to curve upwards, as viewed from the side. Other configurations are also contemplated. In some instances, the second curve 148 may be preset to curve to the left, as viewed proximally to distally along the elongate sheath 140. Other configurations are also contemplated. In some instances, the distal portion 144 and/or the first curve 146 may be configured to bend or deflect in a first direction, wherein the distal tip 142 is bent and/or moved towards and/or closer to the handle 110, toward and/or to a deflected configuration, as shown in FIG. 1. In some cases, the distal portion 144 and/or the first curve 146 may be configured to bend or deflect in a second direction opposite the first direction, wherein the distal tip 142 is bent and/or moved away from and/or farther from the handle 110, toward and/or to a straightened configuration, as shown in FIG. 1. In some cases, the elongate sheath 140 may have only a single curve in the normal or relaxed configuration. In some cases, the elongate sheath 140 may be substantially straight in the normal or relaxed configuration. Other configurations, including combinations of those described herein, are also contemplated.

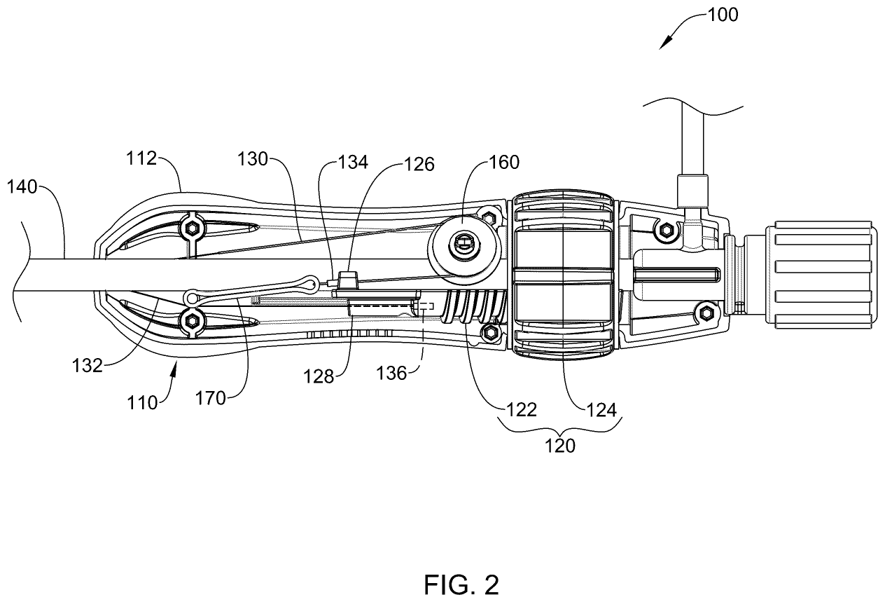

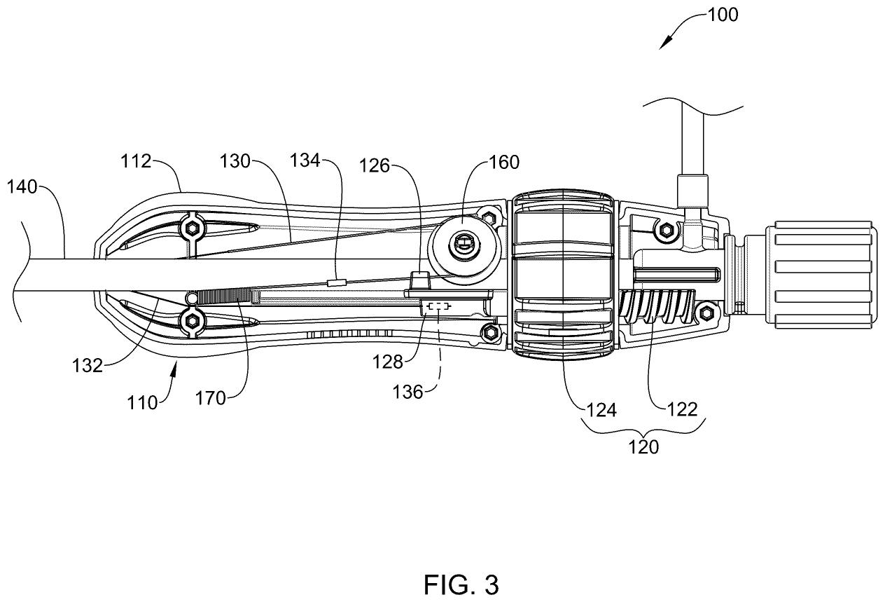

FIGS. 2 and 3 illustrate selected features of the bi-directional steerable catheter 100. In the view shown, a portion of the handle housing 112 has been removed to show internal components of the handle 110. In some cases, the handle 110 may include an axial translation mechanism 120. In some cases, the axial translation mechanism 120 may include a threaded member 122 slidably disposed within the handle 110 and/or the handle housing 112. In some cases, axial translation mechanism 120 may include a rotatable knob 124. In some cases, the rotatable knob 124 may be disposed about and/or may be configured to rotate about, and/or relative to, at least a portion of the handle 110 and/or the handle housing 112. In some cases, the rotatable knob 124 may be configured to engage the threaded member 122 such that rotation of the rotatable knob 124 relative to the handle 110 and/or the handle housing 112 causes axial translation of the threaded member 122 proximally and/or distally within the handle 110 and/or the handle housing 112. In some cases, rotation of the rotatable knob 124 in a clockwise direction, as viewed along the bi-directional steerable catheter 100 proximally to distally, may cause axial translation of the threaded member 122 distally within the handle 110 and/or the handle housing 112. In some cases, rotation of the rotatable knob 124 in a counterclockwise direction, as viewed along the bi-directional steerable catheter 100 proximally to distally, may cause axial translation of the threaded member 122 proximally within the handle 110 and/or the handle housing 112. In some instances, the reverse and/or opposite configuration may be used, wherein clockwise rotation of the rotatable knob 124 moves the threaded member 122 proximally and counterclockwise rotation of the rotatable knob 124 moves the threaded member 122 distally. The orientation of the internal and external threads on the rotatable knob 124 and the threaded member 122, respectively, determine which direction of rotation is tied to which direction of axial translation. Some suitable but non-limiting materials for the axial translation mechanism 120, the threaded member 122, and/or the rotatable knob 124 are described below.

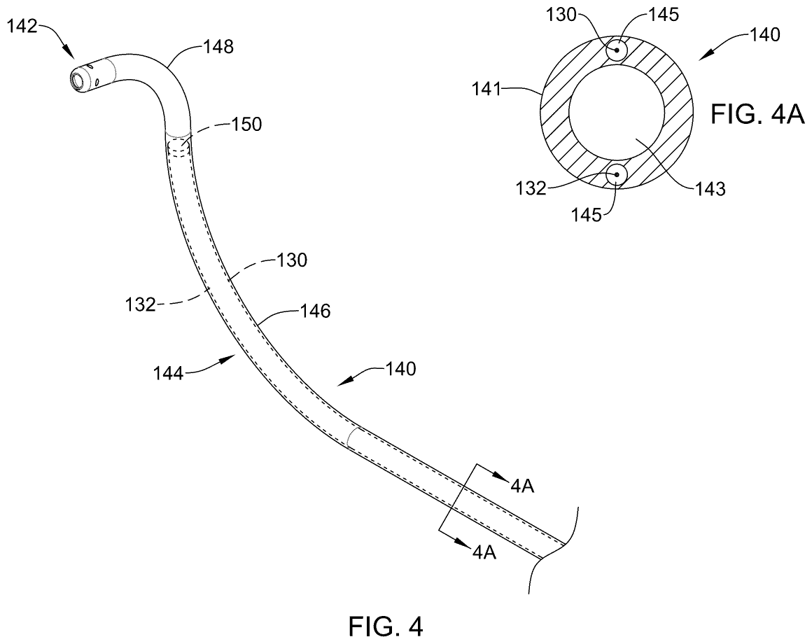

A first steering wire 130 may extend through the elongate sheath 140 from the handle 110 and/or the handle housing 112 to a distal pull ring 150 (e.g., FIG. 4). A second steering wire 132 may extend through the elongate sheath 140 from the handle 110 and/or the handle housing 112 to the distal pull ring 150 (e.g., FIG. 4). The second steering wire 132 may be disposed on an opposite side of the elongate sheath 140 from the first steering wire 130 relative to a central longitudinal axis of the elongate sheath 140. Tension may be applied to the first steering wire 130 and/or the second steering wire 132 as described herein to bend and/or deflect the distal portion 144 and/or the first curve 146 of the elongate sheath 140 (e.g., FIG. 1). The first steering wire 130 may be configured to engage the axial translation mechanism 120 and/or the threaded member 122 to bend and/or deflect the distal portion 144 and/or the first curve 146 of the elongate sheath 140 in the first direction toward the handle 110 and/or the handle housing 112, toward and/or to the deflected configuration (e.g., FIG. 1). The second steering wire 132 may be configured to engage the axial translation mechanism 120 and/or the threaded member 122 to bend and/or deflect the distal portion 144 and/or the first curve 146 of the elongate sheath 140 in the second direction opposite the first direction and away from the handle 110 and/or the handle housing 112, toward and/or to the straightened configuration (e.g., FIG. 1).

In some instances, the bi-directional steerable catheter 100 may include a pulley wheel 160 disposed within the handle 110 and/or the handle housing 112. The pulley wheel 160 may be engaged with the first steering wire 130 via a circumferential channel extending around the pulley wheel 160. In some instances, the pulley wheel 160 may engage the first steering wire 130 at a position proximate a distal end of the threaded member 122. In some cases, the pulley wheel 160 may engage the first steering wire 130 at a position proximal of a distal end of the threaded member 122. In some cases, the bi-directional steerable catheter 100 may include a tensioning member 170. The tensioning member 170 may couple a first end (e.g., a proximal end) of the first steering wire 130 to the handle 110 and/or to the handle housing 112. In at least some instances, the proximal end of the first steering wire 130 may be fixedly coupled to the handle 110 and/or the handle housing 112 by the tensioning member 170. In some instances, the pulley wheel 160 may engage the first steering wire 130 at a position proximal of the tensioning member 170. In some instances, the tensioning member 170 may be coupled to the handle 110 and/or the handle housing 112 at a position distal of the proximal end of the first steering wire 130. In some cases, the tensioning member 170 may be an elastic polymer, as shown in FIG. 2. In another example, the tensioning member 170 may be a coil spring, as shown in FIG. 3. Other configurations are also contemplated. As will be apparent, the tensioning member 170 may be configured to apply a small, non-biasing amount of tension to the first steering wire 130 when the distal portion 144 and/or the first curve 146 of the elongate sheath 140 is disposed in the normal or relaxed configuration and/or when the distal portion 144 and/or the first curve 146 of the elongate sheath 140 is bent and/or deflected in the second direction, toward and/or to the straightened configuration. The purpose of the tensioning member 170 is to prevent the first steering wire 130 from disengaging from the pulley wheel 160 when there is no tension being applied to the first steering wire 130 by the axial translation mechanism 120 and/or the threaded member 122 (e.g., in the normal or relaxed configuration, or toward and/or in the straightened configuration) by holding the first steering wire 130 taught around the pulley wheel 160. Some suitable but non-limiting materials for the pulley wheel 160 and/or the tensioning member 170 are described below.

In addition or alternatively, the bi-directional steerable catheter 100 may include one or more ribs, projections, bosses, or posts extending transversely within the handle housing 112 between opposing walls and/or opposite sides of the handle housing 112. In some instances, the one or more ribs, projections, bosses, or posts may be disposed within the handle housing 112 at positions configured to approximate the diameter and/or the perimeter of the pulley wheel 160. In some instances, the one or more ribs, projections, bosses, or posts may replace the pulley wheel 160. In some embodiments, the one or more ribs, projections, bosses, or posts may be provided in addition to the pulley wheel 160. In some instances, the one or more ribs, projections, bosses, or posts may extend completely across an interior of the handle housing 112 from one side of the handle housing 112 to an opposing side of the handle interior 112. In some cases, the first steering wire 130 may be routed around and/or may slide past the one or more ribs, projections, bosses, or posts in a manner similar to the first steering wire 130 extending around the pulley wheel 160, such that the one or more ribs, projections, bosses, or posts may serve as guides for the first steering wire 130 and prevent loss of motion.

The threaded member 122 may include a first catch 126 extending transversely from the threaded member 122 in a first lateral direction. The first steering wire 130 may extend and/or pass through the first catch 126. The first steering wire 130 may include a first stop element 134 configured to engage with the axial translation mechanism 120 and/or the first catch 126 of the threaded member 122 when the threaded member 122 slides in a distal direction within the handle 110 and/or the handle housing 112 to apply tension to the first steering wire 130, as seen in FIG. 2. The tension applied by the axial translation mechanism 120 and/or the threaded member 122 may be sufficient to overcome the self-bias of the elongate sheath 140 toward the normal or relaxed configuration and bend and/or deflect the distal portion 144 and/or the first curve 146 of the elongate sheath 140 in the first direction.

The threaded member 122 may include a second catch 128 extending transversely from the threaded member 122 in a second lateral direction opposite the first lateral direction. The second steering wire 132 may extend and/or pass through the second catch 128. The second steering wire 132 may include a second stop element 136 configured to engage with the axial translation mechanism 120 and/or the second catch 128 of the threaded member 122 when the threaded member 122 slides in a proximal direction within the handle 110 and/or the handle housing 112 to apply tension to the second steering wire 132, as seen in FIG. 3. The tension applied by the axial translation mechanism 120 and/or the threaded member 122 may be sufficient to overcome the self-bias of the elongate sheath 140 toward the normal or relaxed configuration and bend and/or deflect the distal portion 144 and/or the first curve 146 of the elongate sheath 140 in the second direction.

The pulley wheel 160 permits the threaded member 122 to apply tension to both the first steering wire 130 and the second steering wire 132, depending upon which direction the threaded member 122 is moving. Tension applied to the first steering wire 130 and the second steering wire 132 causes bending and/or deflection of the distal portion 144 and/or the first curve 146 of the elongate sheath 140 away from the normal or relaxed configuration. Since both steering wires extend proximally from the distal pull ring 150, the pulley wheel 160 is needed to reverse the direction of the first steering wire 130 relative to the second steering wire 132 within the handle 110 and/or the handle housing 112 such that the threaded member 122 is able to selectively apply tension to both the first steering wire 130 and the second steering wire 132 by moving in opposite directions. In other configurations, the handle 110 and/or the handle housing 112 may include an internal rib, an internal protrusion, or other features disposed therein, in place of the pulley wheel 160, around which the first steering wire 130 may extend and reverse direction.

When the threaded member 122 is disposed in a central position, the distal portion 144 and/or the first curve 146 of the elongate sheath 140 may be disposed in the normal or relaxed configuration. When the threaded member 122 is disposed in the central position, substantially no tension is being applied to the first steering wire 130 and/or the second steering wire 132. As the threaded member 122 is axially translated proximally and/or distally within the handle 110 and/or the handle housing 112, the threaded member 122 of the axial translation mechanism 120 may engage with the first steering wire 130 and/or the second steering wire 132 to apply tension thereto to bend and/or deflect the distal portion 144 and/or the first curve 146 of the elongate sheath 140 as described herein. Additionally, when the threaded member 122 is disposed in the central position, the first catch 126 may be engaged with the first stop element 134 but tension is not being applied to the first steering wire 130, and the second catch 128 may be engaged with the second stop element 136 but tension is not being applied to the second steering wire 132. As such, the central position of the threaded member 122 may be tension-neutral with respect to the first steering wire 130 and the second steering wire 132.

When the threaded member 122 is moved from the central position toward and/or until disposed in a proximal position, tension may be applied to the second steering wire 132 and the distal portion 144 and/or the first curve 146 of the elongate sheath 140 may be bent and/or deflected in the second direction away from the handle 110 and/or the handle housing 112, or toward and/or to the straightened configuration. In moving the threaded member 122 proximally within the handle 110 and/or the handle housing 112 from the central position, the second catch 128 engages the second stop element 136 and thereafter translates the second stop element 136 proximally, thereby applying tension to the second steering wire 132, as seen in FIG. 3. The first stop element 134 may disengage from the axial translation mechanism 120, the threaded member 122, and/or the first catch 126 to release tension on the first steering wire 130 when the threaded member 122 slides in the proximal direction within the handle 110 and/or the handle housing 112. Accordingly, when the threaded member 122 is moved proximally from the central position, the first catch 126 may be disengaged from the first stop element 134 and the first catch 126 may slide proximally along and/or over the first steering wire 130. The first stop element 134 may be configured to float relative to (e.g., the first stop element 134 may not be directly fixed to) the axial translation mechanism 120, the threaded member 122, and/or the first catch 126 when the threaded member 122 slides in the proximal direction within the handle 110 and/or the handle housing 112. As such, slack would form in the first steering wire 130, which would allow the first steering wire 130 to disengage from the pulley wheel 160, except for the tension applied by the tensioning member 170. The tensioning member 170 holds the first steering wire 130 taught around the pulley wheel 160 while no tension is being applied to the first steering wire 130 by the threaded member 122 and/or the first catch 126. The tensioning member 170 merely absorbs any slack that would be formed in the first steering wire 130 due to the first catch 126 being disengaged from the first stop element 134 and prevents the first steering wire 130 from disengaging from the pulley wheel 160. This feature may be seen in the configuration shown in FIG. 3, for example.

When the threaded member 122 is moved from the central position toward and/or until disposed in a distal position, tension may be applied to the first steering wire 130 and the distal portion 144 and/or the first curve 146 of the elongate sheath 140 may be bent and/or deflected in the first direction toward the handle 110 and/or the handle housing 112, or toward and/or to the deflected configuration. In moving the threaded member 122 distally within the handle 110 and/or the handle housing 112 from the central position, the first catch 126 engages the first stop element 134 and thereafter translates the first stop element 134 distally, thereby applying tension to the first steering wire 130, as seen in FIG. 2. The second stop element 136 may disengage from the axial translation mechanism 120, the threaded member 122, and/or the second catch 128 to release tension on the second steering wire 132 when the threaded member 122 slides in the distal direction within the handle 110 and/or the handle housing 112. Accordingly, when the threaded member 122 is moved distally from the central position, the second catch 128 may be disengaged from the second stop element 136 and the second catch 128 may slide distally along and/or over the second steering wire 132. The second stop clement 136 may be configured to float relative to (e.g., the second stop element 136 may not be directly fixed to) the axial translation mechanism 120, the threaded member 122, and/or the second catch 128 when the threaded member 122 slides in the distal direction within the handle 110 and/or the handle housing 112. As such, slack forms in the second steering wire 132, as may be seen in the configuration shown in FIG. 2, due to the second catch 128 being disengaged from the second stop element 136. As the threaded member 122 is translated distally from the proximal position and/or the central position, the first catch 126 engages the first stop element 134 and the first steering wire 130 is thereafter pulled around the pulley wheel 160 and tension applied by the tensioning member 170 is relieved as tension is instead applied to the first steering wire 130 by the first catch 126 and/or the threaded member 122.

FIG. 4 illustrates features of an example configuration for the elongate sheath 140. In some instances, the elongate sheath 140 may include the soft and/or atraumatic distal tip 142. In some instances, the elongate sheath 140 may include the distal portion 144 having the first curve 146 and the second curve 148, such that the elongate sheath 140 has a preset double curve, in the normal or relaxed configuration. In some embodiments, the first curve 146 may be preset to curve upwards, as viewed from the side. Other configurations are also contemplated. In some cases, the second curve 148 may be preset to curve to the left, as viewed proximally to distally along the elongate sheath 140. Other configurations are also contemplated.

In some instances, the elongate sheath 140 may include a wall 141 defining a central lumen 143 extending from a proximal end to the distal tip 142 along the central longitudinal axis of the elongate sheath 140. In at least some cases, the central lumen 143 may be coaxial with the central longitudinal axis of the elongate sheath 140. In some cases, the central lumen 143 may be a guidewire lumen. In some cases, the central lumen 143 may be a device lumen used to deliver a medical device or implant. In some cases, the central lumen 143 may have multiple uses. The elongate sheath 140 may include a plurality of steering wire lumens 145 extending and/or disposed within the wall 141. In some embodiments, the plurality of steering wire lumens 145 may include a first steering wire lumen and a second steering wire lumen. In some instances, the plurality of steering wire lumens 145 may include more than two steering wire lumens. In some embodiments, the plurality of steering wire lumens 145 may be oriented substantially parallel to the central lumen 143 and/or the central longitudinal axis of the elongate sheath 140. In some instances, the plurality of steering wire lumens 145 may be disposed opposite each other and/or on opposite sides of the elongate sheath 140 relative to the central lumen 143 and/or the central longitudinal axis of the elongate sheath 140. Other configurations are also contemplated.

As discussed herein, a distal pull ring 150 may be disposed within the distal portion 144 of the elongate sheath 140. In some instances, the distal pull ring 150 may be disposed proximal to the second curve 148 and/or the distal tip 142. In at least some instances, the distal pull ring 150 may be disposed proximate a distal end of the first curve 146. In some cases, the distal pull ring 150 may be embedded within the wall 141 of the elongate sheath 140. In some cases, the distal pull ring 150 may be secured, bonded, and/or fixedly attached to an inner surface of the wall 141 of the elongate sheath 140. Other configurations are also contemplated. Some suitable but non-limiting materials for the distal pull ring 150 are described below.

The first steering wire 130 and the second steering wire 132 may each be slidably disposed within the plurality of steering wire lumens 145. In one example, the first steering wire 130 may be slidably disposed within the first steering wire lumen and the second steering wire 132 may be disposed within the second steering wire lumen. The first steering wire 130 and the second steering wire 132 may be fixedly attached (e.g., bonded, welded, etc.) to the distal pull ring 150. For example, a distal end of the first steering wire 130 may be fixedly attached to the distal pull ring 150 and a distal end of the second steering wire 132 may be fixedly attached to the distal pull ring 150 at a position opposite the distal end of the first steering wire 130 relative to the central longitudinal axis of the elongate sheath 140. Some suitable but non-limiting materials for the first steering wire 130 and the second steering wire 132 are described below.

In some instances, the elongate sheath 140 may be sized in accordance with its intended use. For example, the elongate sheath 140 can have a length that is in the range of about 50 to about 200 centimeters, about 75 to about 175 centimeters, or about 100 to about 150 centimeters. Other lengths are also contemplated. It is further contemplated that the outer diameter of the elongate sheath 140 may vary based on the use or application. In some examples, the outer diameter of the elongate sheath 140 may be about 2 millimeters (mm), about 3 mm (or 9 French), about 3.5 mm, about 4 mm (or 12 French), about 4.5 mm, about 5 mm (or 15 French), about 5.33 mm, about 5.5 mm, about 5.66 mm (or 17 French), about 6 mm, about 6.5 mm, about 7 mm (or 21 French), about 8 mm, or other suitable sizes. In some embodiments, the outer diameter of the elongate sheath 140 may be a maximum of 5.66 mm (17 French), and may be smaller than 5.66 mm (17 French). Other configurations are also contemplated. Some suitable but non-limiting materials for the elongate sheath 140 are described below.

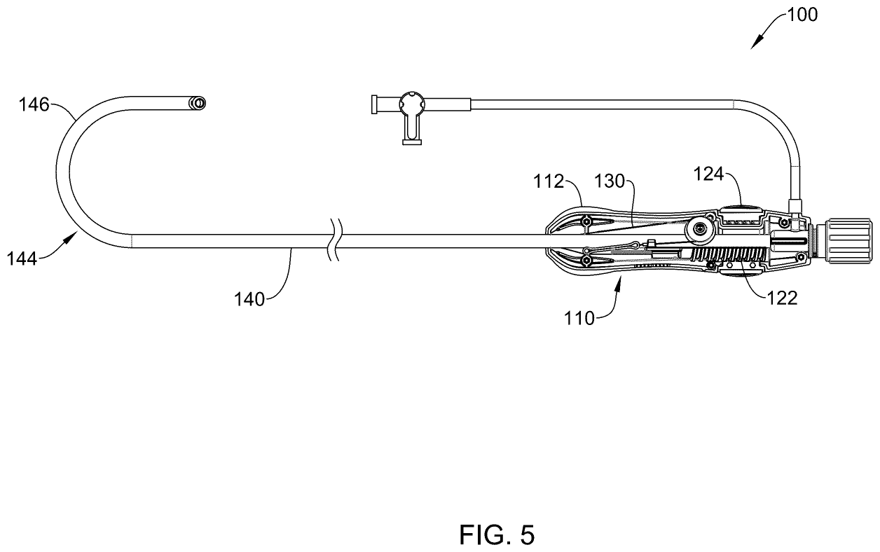

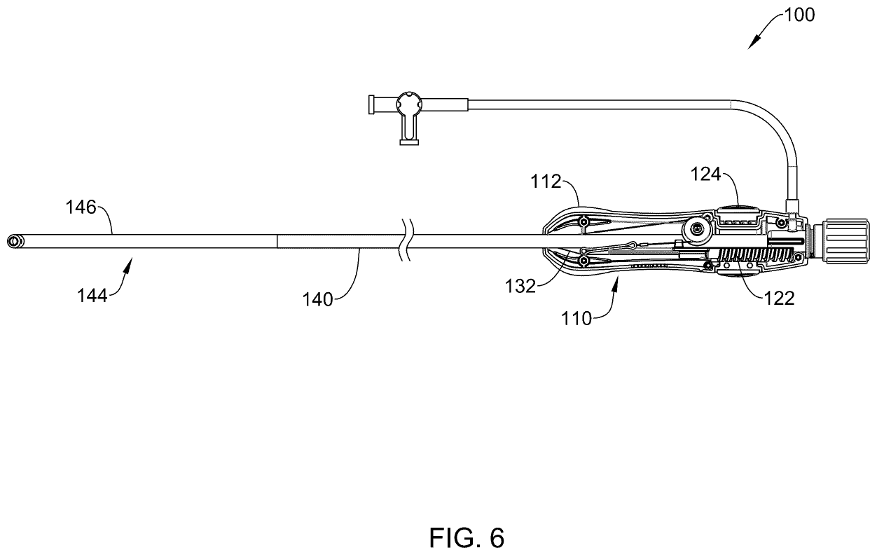

FIGS. 5 and 6 illustrate the relationship between certain features of the bi-directional steerable catheter 100 in the deflected and straightened configurations. As seen in FIG. 5, clockwise rotation of the rotatable knob 124, as viewed proximally to distally, has moved the threaded member 122 distally within the handle 110 and/or the handle housing 112, thereby applying tension to the first steering wire 130 and bending or deflecting the distal portion 144 and/or the first curve 146 of the elongate sheath 140 toward the handle 110 and/or the handle housing 112, or toward and/or to the deflected configuration. As seen in FIG. 6, counterclockwise rotation of the rotatable knob 124, as viewed proximally to distally, has moved the threaded member 122 proximally within the handle 110 and/or the handle housing 112, thereby applying tension to the second steering wire 132 and bending or deflecting the distal portion 144 and/or the first curve 146 of the elongate sheath 140 away from the handle 110 and/or the handle housing 112, or toward and/or to the straightened configuration. As discussed herein, other configurations are also contemplated.

In some instances, the bi-directional steerable catheter 100 may include an auto lock feature, meaning that the rotatable knob 124 may be rotated to a particular position resulting in the distal portion 144 of the elongate sheath 140 being bent or deflected to a particular curved shape, and then the rotatable knob 124 may be released by the user without the rotatable knob 124 moving on its own as a result of any forces being applied to the rotatable knob 124 by the stresses induced within the elongate sheath 140. In some cases, the auto lock feature may be achieved through one or features of the bi-directional steerable catheter 100 without requiring any additional components. In some cases, the auto lock feature may be achieved without requiring any additional steps to lock or unlock the rotatable knob 124, for example. In some instances, the auto lock feature may provide a tactile neutral position that allows the user to know where the start position is by feel. In some cases, the tactile neutral position may correspond to a load on the first steering wire 130 is equal in magnitude to a load on the second steering wire 132. In some instances, the tactile neutral position may correspond to no load on the first steering wire 130 and no load on the second steering wire 132. In some cases, this may correspond to a tension-neutral central position of the threaded member 122.

In some instances, inclusion of particular surface characteristics of parts of the rotatable knob 124 and the handle housing 112 may help to facilitate an auto lock feature. In some instances, particular geometric relationships between the rotatable knob 124 and the handle housing 112 may help to facilitate an auto lock feature. In some instances, particular material selections for parts of the rotatable knob 124 and the handle housing 112 may help to facilitate an auto lock feature. In some cases, combinations of surface characteristics, geometric relationships, and material selections may together help to facilitate an auto lock feature.

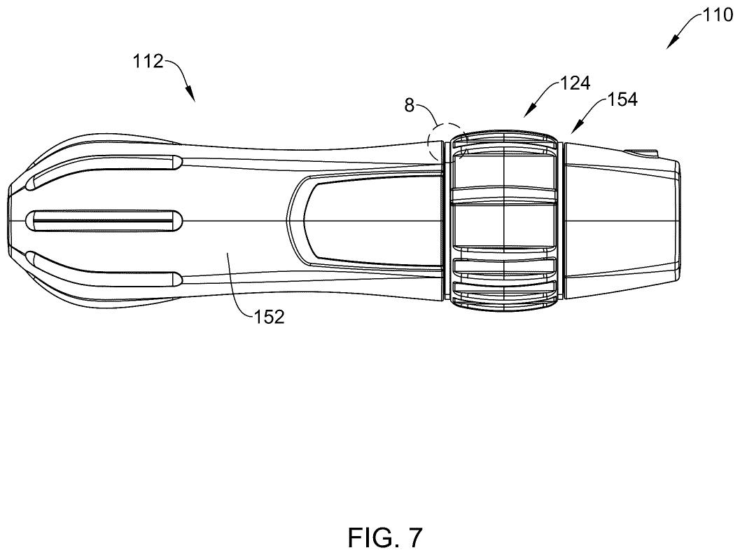

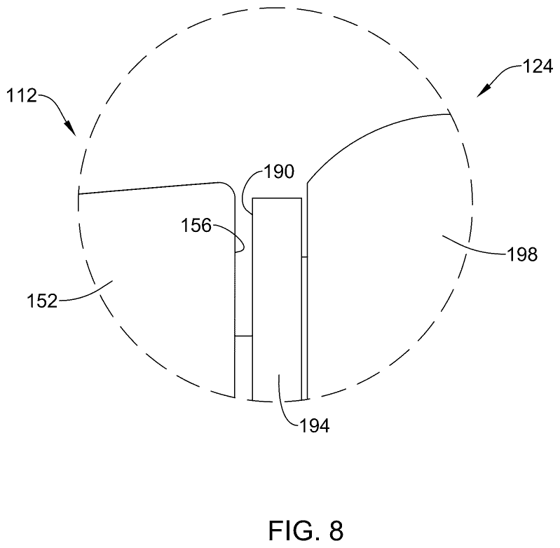

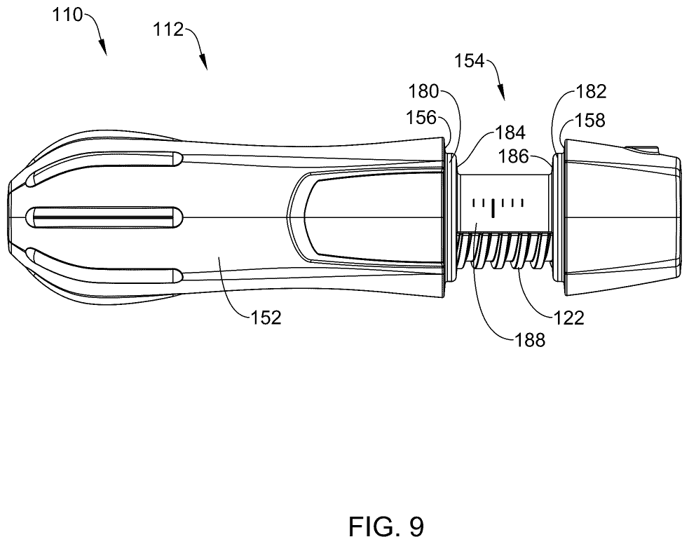

FIG. 7 shows the handle 110, including the handle housing 112 and the rotatable knob 124 and FIG. 8 is an enlarged view of a portion of FIG. 7. FIG. 9 shows some features of the handle 110 with the rotatable knob 124 removed to show particular geometric features of the handle housing 112. The handle housing 112 includes a graspable outer surface 152 and a handle recess 154. In some cases, the rotatable knob 124 may be considered as being disposed within the handle recess 154. The handle recess 154 may include a distal annular bearing surface 156 and a proximal annular bearing surface 158. In some instances, the distal annular bearing surface 156 and the proximal annular bearing surface 158 may engage corresponding surfaces on the rotatable knob 124.

In some cases, the distal annular bearing surface 156 and the proximal annular bearing surface 158 may be adapted to have particular surface characteristics that can contribute to achieving an auto lock feature. In some cases, the distal annular bearing surface 156 and the proximal annular bearing surface 158 may each have a particular surface finish. As an example, the distal annular bearing surface 156 and the proximal annular bearing surface 158 may each have an average surface roughness Sa that is less than about 1.27 micrometers (50 microinches). As another example, the distal annular bearing surface 156 and the proximal annular bearing surface 158 may each have an average surface roughness Sa that is in a range of about 0.127 micrometers (5 micro inches) to about 1.02 micrometers (40 microinches), or any value therebetween. Average surface roughness Sa may be defined as an arithmetic average of the absolute values of the measured height deviations from the mean surface, taken within an evaluation area. In general, the higher the average surface roughness Sa, the rougher the surface may be considered to be. The lower the average surface roughness Sa, the smoother the surface may be considered to be.

In some cases, all of the handle recess 154 may have a similar average surface roughness Sa. For example, each of a first radial surface 180 and a second radial surface 182 may have an average surface roughness Sa that is similar or even identical to the average surface roughness Sa of the distal annular bearing surface 156 and the proximal annular bearing surface 158. In some cases, each of a first inner bearing surface 184 and a second inner bearing surface 186 may have an average surface roughness Sa that is similar or even identical to the average surface roughness Sa of the distal annular bearing surface 156 and the proximal annular bearing surface 158. In some cases, an inner profile 188 may also have an average surface roughness Sa that is similar or even identical to the average surface roughness Sa of the distal annular bearing surface 156 and the proximal annular bearing surface 158. In some cases, the graspable surface 152 of the handle housing 112 may have a surface that is rougher than that of the handle recess 154. As an example, the graspable surface 152 of the handle housing 112 may have an average surface roughness Sa that is greater than about 1.91 micrometers (75 microinches), or perhaps greater than about 2.54 micrometers (100 microinches) or even about 3.81 micrometers (150 microinches) in order to make it easier for a user to grasp and hold onto the handle housing 112.

In some cases, the distal annular bearing surface 156 and the proximal annular bearing surface 158 may be formed of particular materials that can contribute to achieving an auto lock feature. In some cases, the distal annular bearing surface 156 and the proximal annular bearing surface 158 may be formed of a polymer blend such as a polycarbonate acrylonitrile-butadiene-styrene polymer blend. Suitable examples of polycarbonate acrylonitrile-butadiene-styrene polymer blends are available commercially from SABIC under the CYCOLOY™ tradename. In some cases, the entire handle housing 112 may be formed of a polycarbonate acrylonitrile-butadiene-styrene polymer blend such as those available commercially from SABIC under the CYCOLOY™ tradename. In some cases, the entire handle housing 112 may be molded within a mold that is specifically treated or processed to provide the desired surface characteristics such as surface roughness to both the graspable surface 152 and the handle recess 154.

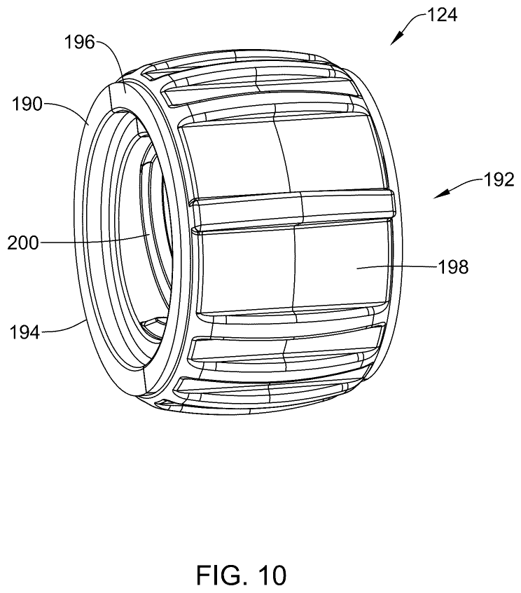

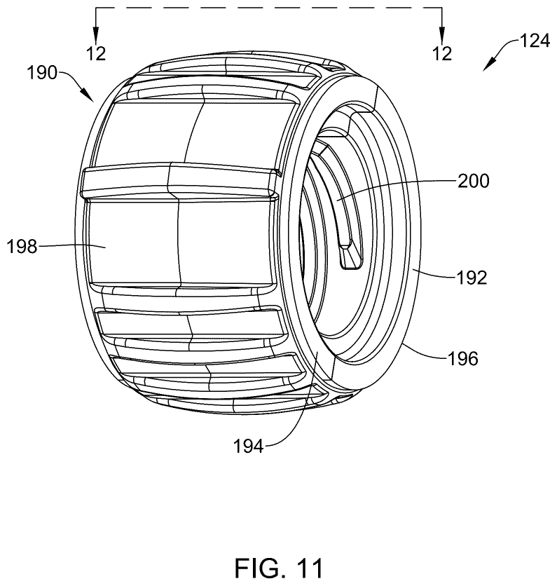

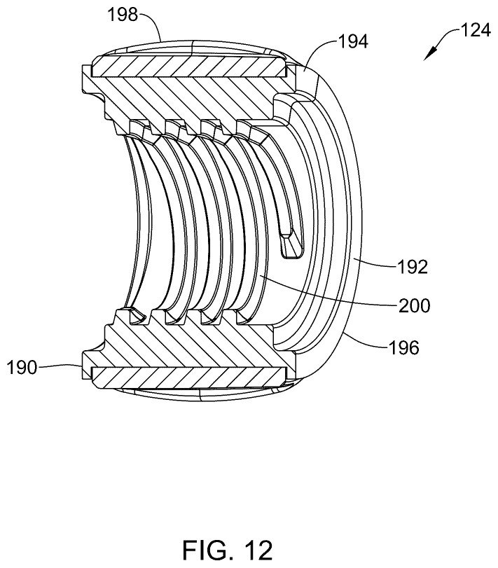

FIGS. 10 and 11 show first and second views of the rotatable knob 124, and show particular geometric features of the rotatable knob 124 and the components thereof. FIG. 12 is a cross-sectional view of the rotatable knob 124, taken along the line 12-12 of FIG. 11. In some cases, the rotatable knob 124 includes a distal annular bearing surface 190 and a proximal annular bearing surface 192. In some cases, the distal annular bearing surface 190 may be at least substantially parallel to the distal annular bearing surface 156 of the handle recess 154 and/or the proximal annular bearing surface 192 may be at least substantially parallel with the proximal annular bearing surface 158 of the handle recess 154. In this, “substantially parallel” may be defined as being within ten percent of being parallel, or perhaps within five percent or less of parallel.

In some cases, the rotatable knob 124 may translate a small amount in either a distal direction or a proximal direction, depending on which direction the rotatable knob 124 is being rotated, and correspondingly which direction the distal portion 144 of the distal sheath 140 is being bent and the resulting forces being transmitted back to the handle 110 by the first steering wire 130 and/or the second steering wire 132. Having these contacting surfaces, such as the distal annular bearing surface 156 and the distal annular bearing surface 190 or the proximal annular bearing surface 158 and the proximal annular bearing surface 192, parallel to each can increase the relative contact forces therebetween. The frictional forces generated between the distal annular bearing surface 156 and the distal annular bearing surface 190 or the proximal annular bearing surface 158 and the proximal annular bearing surface 192 help to facilitate an auto lock feature.

In some cases, the distal annular bearing surface 190 and the proximal annular bearing surface 192 may be adapted to have particular surface characteristics that can contribute to achieving an auto lock feature. In some cases, the distal annular bearing surface 190 and the proximal annular bearing surface 192 may each have a particular surface finish. As an example, the distal annular bearing surface 190 and the proximal annular bearing surface 192 may each have an average surface roughness Sa that is less than about 1.27 micrometers (50 microinches). As another example, the distal annular bearing surface 190 and the proximal annular bearing surface 192 may each have an average surface roughness Sa that is in a range of about 0.127 micrometers (5 microinches) to about 1.02 micrometers (40 microinches), or any value therebetween.

In some cases, material choices for the rotatable knob 124, or portions thereof, may also contribute to an auto lock feature. In some instances, the rotatable knob 124 may include an inner component that interacts with the threaded member 122 and an outer component that is adapted to allow a user to easily grasp and rotate the rotatable knob 124. In some cases, the rotatable knob 124 may include a first dial half 194 and a second dial half 196. As an example, the first dial half 194 and the second dial half 196 may fit together with a snap-fit. As another example, the first dial half 194 and the second dial half 196 may be adhesively secured together, or may be secured together via one or more fasteners such as screws. In some cases, the first dial half 194 and the second dial half 196 are each formed from a polycarbonate acrylonitrile-butadiene-styrene polymer blend that includes a small amount of polytetrafluoroethylene. As an example, the first dial half 194 and the second dial half 196 may each be formed from a polycarbonate acrylonitrile-butadiene-styrene polymer blend that includes about five weight percent of polytetrafluoroethylene. A graspable boot 198 extends around the first dial half 194 and the second dial half 196 and makes the rotatable knob 124 easily graspable for a user. As an example, the graspable boot 198 may be formed of a resilient material such as a rubber or an elastomeric polymer.

In some cases, the rotatable knob 124 has a threaded inner surface 200 that is adapted to engage the threaded member 202. In some cases, the first dial half 194 may form a first part of the threaded inner surface 200 and the second dial half 196 may form a second part of the threaded inner surface 200. In some cases, the threaded inner surface 200 may have an average surface roughness Sa that is less than about 1.27 micrometers (50 microinches). In some cases, the threaded inner surface 200 may have an average surface roughness Sa that is in a range of about 0.127 micrometers (5 microinches) to about 1.02 micrometers (40 microinches), or any intervening value therebetween.

The materials that can be used for the various components of the bi-directional steerable catheter (and/or other systems or components disclosed herein) and the various elements thereof disclosed herein may include those commonly associated with medical devices. For simplicity purposes, the following discussion makes reference to the sheath, etc. However, this is not intended to limit the devices and methods described herein, as the discussion may be applied to other elements, members, components, or devices disclosed herein, such as, but not limited to, the elongate sheath, the handle, the handle housing, the threaded member(s), the carriage member(s), the steering wire(s), etc. and/or elements or components thereof.

In some embodiments, the bi-directional steerable catheter and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 444V, 444L, and 314LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R44035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R44003 such as ELGILOY®, PHYNOX®, and the like); platinum enriched stainless steel; titanium; platinum; palladium; gold; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear than the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the bi-directional steerable catheter and/or components thereof, may also be doped with, made of, or otherwise include a radiopaque material. Radiopaque materials are understood to be materials capable of producing a relatively bright image on a fluoroscopy screen or another imaging technique during a medical procedure. This relatively bright image aids a user in determining the location of the bi-directional steerable catheter. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the bi-directional steerable catheter to achieve the same result.

In some embodiments, a degree of Magnetic Resonance Imaging (MRI) compatibility is imparted into the bi-directional steerable catheter. For example, the bi-directional steerable catheter and/or components or portions thereof, may be made of a material that does not substantially distort the image and create substantial artifacts (e.g., gaps in the image). Certain ferromagnetic materials, for example, may not be suitable because they may create artifacts in an MRI image. The bi-directional steerable catheter or portions thereof, may also be made from a material that the MRI machine can image. Some materials that exhibit these characteristics include, for example, tungsten, cobalt-chromium-molybdenum alloys (e.g., UNS: R44003 such as ELGILOY®, PHYNOX®, and the like), nickel-cobalt-chromium-molybdenum alloys (e.g., UNS: R44035 such as MP35-NR and the like), nitinol, and the like, and others.

In some embodiments, the bi-directional steerable catheter and/or portions thereof, may be made from or include a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, polyurethane silicone copolymers (for example, ElastEon® from Aortech Biomaterials or ChronoSil® from AdvanSource Biomaterials), biocompatible polymers, other suitable materials, or mixtures, combinations, copolymers thereof, polymer/metal composites, and the like. In some embodiments, the sheath can be blended with a liquid crystal polymer (LCP). For example, the mixture can contain up to about 6 percent LCP.

In some embodiments, the bi-directional steerable catheter may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni—Co—Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the bi-directional steerable catheter may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethylketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl keton, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The disclosure's scope is, of course, defined in the language in which the appended claims are expressed.

What is claimed is:

1. A bi-directional steerable catheter, comprising:
a handle including an outer surface and a handle recess formed within the outer surface;
an axial translation mechanism disposed within the handle, the axial translation mechanism including:
a threaded member slidably disposed within the handle; and
a rotatable knob disposed about the handle recess and configured to engage the threaded member such that rotation of the rotatable knob relative to the handle causes axial translation of the threaded member within the handle;
an elongate sheath extending distally from the handle;
a first steering wire extending through the elongate sheath from the handle to a distal pull ring and configured to engage with the threaded member to bend a distal portion of the elongate sheath in a first direction;
a second steering wire extending through the elongate sheath from the handle to the distal pull ring and configured to engage with the threaded member to bend the distal portion of the elongate sheath in a second direction opposite the first direction;
wherein the bi-directional steerable catheter is adapted to provide an auto lock feature that holds the rotatable knob at its rotational position when a user releases the rotatable knob while the distal portion of the elongate sheath is bent in the first direction or in the second direction.

2. The bi-directional steering catheter of claim 1, wherein:
the rotatable knob defines:
a first distal annular bearing surface; and
a first proximal annular bearing surface; and
the handle recess defines:
a second distal annular bearing surface that is parallel to and adapted to engage the first distal annular bearing surface; and
a second proximal annular surface bearing surface that is parallel to and adapted to engage the first proximal annular bearing surface.

3. The bi-directional steering catheter of claim 2, wherein:
the first distal annular bearing surface has an average surface roughness of less than about 1.27 micrometers (50 microinches);
the first proximal annular bearing surface has an average surface roughness of less than about 1.27 micrometers (50 microinches);
the second distal annular bearing surface has an average surface roughness of less than about 1.27 micrometers (50 microinches); and
the second proximal annular bearing surface has an average surface roughness of less than about 1.27 micrometers (50 microinches).

4. The bi-directional steering catheter of claim 2, wherein:
the first distal annular bearing surface has an average surface roughness that is in a range of about 0.127 micrometers (5 microinches) to about 1.02 micrometers (40 microinches);
the first proximal annular bearing surface has an average surface roughness that is in a range of about 0.127 micrometers (5 microinches) to about 1.02 micrometers (40 microinches);
the second distal annular bearing surface has an average surface roughness that is in a range of about 0.127 micrometers (5 microinches) to about 1.02 micrometers (40 microinches); and
the second proximal annular bearing surface has an average surface roughness that is in a range of about 0.127 micrometers (5 microinches) to about 1.02 micrometers (40 microinches).

5. The bi-directional steering catheter of claim 1, wherein the rotatable knob has a threaded inner surface adapted to engage the threaded member, and the threaded inner surface has an average surface roughness of less than about 1.27 micrometers (50 microinches).

6. The bi-directional steering catheter of claim 5, wherein the threaded inner surface has an average surface roughness that is in a range of about 0.127 micrometers (5 microinches) to about 1.02 micrometers (40 microinches).

7. The bi-directional steering catheter of claim 1, wherein the handle comprises a polycarbonate acrylonitrile-butadiene-styrene polymer blend.

8. The bi-directional steering catheter of claim 1, wherein the rotatable knob comprises:
a first dial half defining part of the threaded inner surface;
a second dial half defining part of the threaded inner surface and secured to the first dial half; and
a graspable boot disposed over the first dial half and the second dial half.

9. The bi-directional steering catheter of claim 8, wherein the first dial half and the second dial half each comprise a polycarbonate acrylonitrile-butadiene-styrene polymer blend that includes about five weight percent poly tetrafluoroethylene.

10. The bi-directional steering catheter of claim 1, wherein the auto lock feature includes a tactile neutral position when a load on the first steering wire is equal to a load on the second steering wire.

11. A bi-directional steerable catheter, comprising:
a handle including an outer surface and a handle recess formed within the outer surface, the handle recess having an average surface roughness of less than about 1.27 micrometers (50 microinches);
an axial translation mechanism disposed within the handle, the axial translation mechanism including:
a threaded member slidably disposed within the handle; and
a rotatable knob disposed about the handle recess and configured to engage the threaded member such that rotation of the rotatable knob relative to the handle causes axial translation of the threaded member within the handle;
wherein portions of the rotatable knob contacting the handle recess have an average surface roughness of less than about 1.27 micrometers (50 microinches);
an elongate sheath extending distally from the handle;
a first steering wire extending through the elongate sheath from the handle to a distal pull ring and configured to engage with the threaded member to bend a distal portion of the elongate sheath in a first direction;
a second steering wire extending through the elongate sheath from the handle to the distal pull ring and configured to engage with the threaded member to bend the distal portion of the elongate sheath in a second direction opposite the first direction.

12. The bi-directional steering catheter of claim 11, wherein:
the portions of the rotatable knob contacting the handle recess include:
a first distal annular bearing surface; and
a first proximal annular bearing surface; and
the handle recess includes:
a second distal annular bearing surface that is parallel to and adapted to engage the first distal annular bearing surface; and
a second proximal annular surface bearing surface that is parallel to and adapted to engage the first proximal annular bearing surface.

13. The bi-directional steering catheter of claim 11, wherein the rotatable knob has a threaded inner surface adapted to engage the threaded member, and the threaded inner surface has an average surface roughness of less than about 1.27 micrometers (50 microinches).

14. The bi-directional steering catheter of claim 11, wherein the handle comprises a polycarbonate acrylonitrile-butadiene-styrene polymer blend.

15. The bi-directional steering catheter of claim 11, wherein the rotatable knob comprises:
a first dial half defining part of the threaded inner surface;
a second dial half defining part of the threaded inner surface and secured to the first dial half; and
a graspable boot disposed over the first dial half and the second dial half.

16. The bi-directional steering catheter of claim 15, wherein the first dial half and the second dial half each comprise a polycarbonate acrylonitrile-butadiene-styrene polymer blend that includes about five weight percent poly tetrafluoroethylene.

17. A bi-directional steerable catheter, comprising:
a handle including an outer surface and a handle recess formed within the outer surface;
an axial translation mechanism disposed within the handle, the axial translation mechanism including:
a threaded member slidably disposed within the handle; and
a rotatable knob disposed about the handle recess and configured to engage the threaded member such that rotation of the rotatable knob relative to the handle causes axial translation of the threaded member within the handle, the rotatable knob including a first dial half and a second dial half that together define:
a first distal annular bearing surface;
a first proximal annular bearing surface; and
a threaded surface adapted to engage the threaded member;
the handle recess including:
a second distal annular bearing surface that is parallel to and adapted to engage the first distal annular bearing surface; and
a second proximal annular surface bearing surface that is parallel to and adapted to engage the first proximal annular bearing surface
an elongate sheath extending distally from the handle;
a first steering wire extending through the elongate sheath from the handle to a distal pull ring and configured to engage with the threaded member to bend a distal portion of the elongate sheath in a first direction; and
a second steering wire extending through the elongate sheath from the handle to the distal pull ring and configured to engage with the threaded member to bend the distal portion of the elongate sheath in a second direction opposite the first direction;
wherein the handle comprises a polycarbonate acrylonitrile-butadiene-styrene polymer blend; and
wherein the first dial half and the second dial half each comprise a polycarbonate acrylonitrile-butadiene-styrene polymer blend that includes about five weight percent poly tetrafluoroethylene.

18. The bi-directional steering catheter of claim 17, wherein:
the first distal annular bearing surface has an average surface roughness of less than about 1.27 micrometers (50 microinches);
the first proximal annular bearing surface has an average surface roughness of less than about 1.27 micrometers (50 microinches);
the second distal annular bearing surface has an average surface roughness of less than about 1.27 micrometers (50 microinches); and
the second proximal annular bearing surface has an average surface roughness of less than about 1.27 micrometers (50 microinches).

19. The bi-directional steering catheter of claim 17, wherein:
the first distal annular bearing surface has an average surface roughness that is in a range of about 0.127 micrometers (5 microinches) to about 1.02 micrometers (40 microinches);
the first proximal annular bearing surface has an average surface roughness that is in a range of about 0.127 micrometers (5 microinches) to about 1.02 micrometers (40 microinches);
the second distal annular bearing surface has an average surface roughness that is in a range of about 0.127 micrometers (5 microinches) to about 1.02 micrometers (40 microinches); and
the second proximal annular bearing surface has an average surface roughness that is in a range of about 0.127 micrometers (5 microinches) to about 1.02 micrometers (40 microinches).

20. The bi-directional steering catheter of claim 17, wherein the threaded inner surface has an average surface roughness that is in a range of about 0.127 micrometers (5 microinches) to about 1.02 micrometers (40 microinches).
